(19) United States
(12) Patent Application Publication
(10) Pub. No.: US 2005/0272981 A1
(43) Pub. Date: Dec. 8, 2005

(54) Threaded fulcrum clamp

(75) Inventors: Todd M. Bjork, River Falls, WI (US); Todd William Sharratt, Stillwater, MN (US); Christopher L. Berg, Crystal, MN (US)

Correspondence Address: Z. Peter Sawicki;Westman, Champlin & Kelly, Suite 1600, 900 Second Avenue South, Minneapolis, MN, 55402-3319, US

(73) Assignee: Minnesota Scientific, Inc., St. Paul, MN (US)

(21) Appl. No.: 11/034,231

(22) Filed: Jan. 12, 2005

Related U.S. Application Data
(63) Continuation-in-part of application No. 10/732,491, filed on Dec. 10, 2003.
Application No. 10/732,491 is a continuation of application No. 10/664,195, filed on Sep. 17, 2003.
(60) Provisional application No. 60/535,910, filed on Jan. 12, 2004.

(51) Int. Cl. A61B 1/32

(52) U.S. Cl. 600/227

(57) ABSTRACT

A surgical clamp includes a first clamping member comprising a first clamping surface for engaging a first surgical rod and an attachment end. A second clamping member is mounted with respect to the first clamping member wherein the second clamping member comprises a second clamping surface for engaging a second surgical rod. A shaft is disposed through and positioned within the attachment end of the first clamping member and wherein the shaft is in communication with the second clamping member. A force providing mechanism is disposed between the first and second clamping members and disposed about the shaft wherein when the force providing mechanism is positionable between a first position with the first and second clamping members being in a nonclamping position and a second position with the first and second clamping members being in a clamping position wherein the first and second sockets constrict to retain the first and second surgical rods in selected positions.

### CROSS REFERENCE TO RELATED APPLICATIONS

This application claims priority of U.S. Provisional Application No. 60/535,910, filed on Jan. 12, 2004, the content of which is hereby incorporated by reference in its entirety.

This application is also a continuation-in-part of application Ser. No. 10/732,491 filed on Dec. 10, 2003, the content of which is hereby incorporated by reference in its entirety, which is a continuation of application Ser. No. 10/664,195, filed Sep. 17, 2003, the contents of which is hereby incorporated by reference in its entirety.

### BACKGROUND OF THE INVENTION

The present invention relates generally to a captivated clamp for use in mounting surgical retractors with respect to an operating table. More particularly, the present invention relates to a clamp that is positioned into a clamping position with a threaded actuating mechanism flexing a fulcrum portion.

Prior to performing a surgical procedure requiring retraction, a retractor support apparatus is typically positioned about the surgical site. Retractor clamps are commonly used to mount the retractor support apparatus with respect to an operating table, as well as to attach surgical retractors to the retractor support apparatus.

A retractor clamp typically includes a first clamping member, a second clamping member and a handle. A typical configuration for a clamping member is to have the clamping member fabricated from a unitary structure that is generally in the shape of the letter “U”. In each of the retractor clamps, the object to be clamped is placed between the legs of the U-shaped structure so that the object is proximate the base of the U-shaped structure. Movement of the legs of the U-shaped structure towards each other causes the object to be clamped with respect to the clamping member.

However, the use U-shaped clamping structures may be cumbersome in certain surgical procedures. First, the U-shaped clamping structure must be positioned over an end of a retractor support apparatus and slid into a desired position. When previously placed retractor clamps are disposed between the end of the support arm and the desired location, the interfering clamps must be removed to allow the additional surgical clamp to be disposed in the desired location. Having to disassemble at least a portion of the surgical support apparatus about a surgical site to add additional retractors adds unnecessary additional time and expense to the surgical procedure. Additionally, an end of the retractor handle must be positioned through the other U-shaped clamps prior to positioning the retractor in the retracting position.

### SUMMARY OF THE INVENTION

The present invention includes a clamp for clamping two elongated members where each elongated member has a central axis. The clamp includes at least two clamping members both of which have clamping sockets with an opening for accepting an elongated member in a generally orthogonal direction to the axis of the member. The clamp also includes a mechanism that constricts the opening of the clamping socket of both members generally simultaneously such that the respective elongated member is captivated within each clamping socket.

The present invention also includes a surgical clamp having a clamping portion with first and second legs that are spaced apart and extend from different sides of the clamping portion. A shaft extends through the first and second legs and has an actuating mechanism threadably engaging the shaft between the first and second legs. The actuating mechanism is movable to force the first and second legs to move in opposing directions thereby placing the clamping portion into a clamping position.

The present invention also includes a surgical clamp having a first clamping member comprising a first clamping surface for engaging a first surgical rod and an attachment end. A second clamping member is mounted with respect to the first clamping member wherein the second clamping member comprises a second clamping surface for engaging a second surgical rod. A shaft is disposed through and positioned within the attachment end of the first clamping member and wherein the shaft is in communication with the second clamping member. A force providing mechanism is disposed between the first and second clamping members and disposed about the shaft wherein when the force providing mechanism is positionable between a first position with the first and second clamping members being in a nonclamping position and a second position with the first and second clamping members being in a clamping position wherein the first and second sockets constrict to retain the first and second surgical rods in selected positions.

### BRIEF DESCRIPTION OF THE DRAWINGS

FIG. 1 is a perspective view of the surgical clamp of the present invention in a non-clamping position.

FIG. 2 is another perspective view of the surgical clamp of the present invention in a non-clamping position.

FIG. 3 is an exploded view of the surgical clamp of the present invention.

FIG. 4 is a sectional view of the surgical clamp of the present invention in a clamping position.

### DETAILED DESCRIPTION OF THE PREFERRED EMBODIMENTS

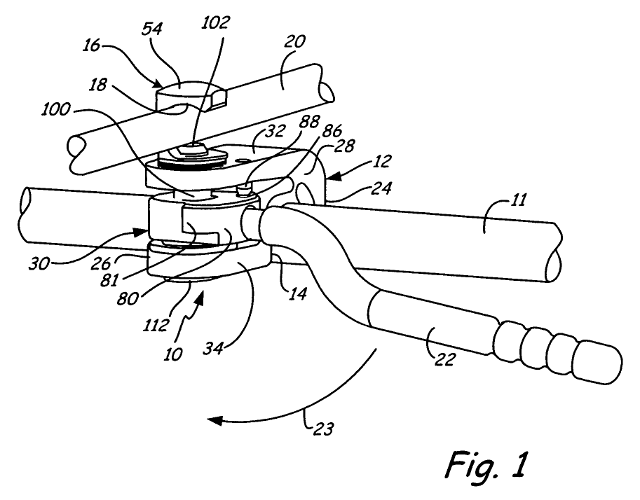

The present invention includes a low-profile surgical clamp generally illustrated at FIG. 1 at 10. The clamp is particularly suited for mounting a retractor (not shown) with respect to an operating table (not shown). The clamp 10 includes a bottom clamping member 12 having a support arm clamping surface 14 proximate a clamping end or portion 24 and a top clamping member 16 having a retractor clamping surface 18. Other components may be substituted for the top clamping member 16 such as, but not limited to, permanently attached retractors, or retractors that are attached using other types of clamps or fasteners.

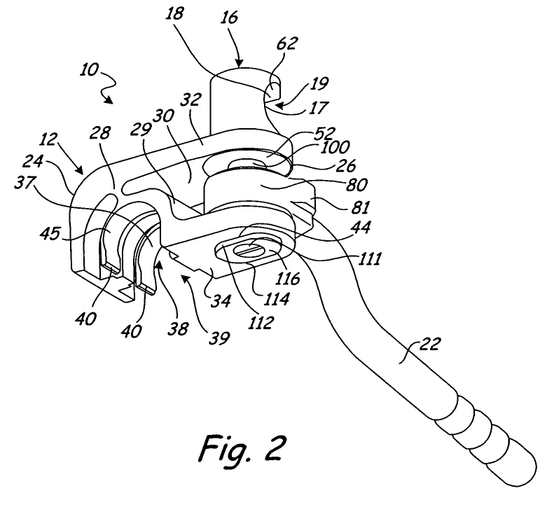

Referring to FIGS. 2-4, the bottom clamping member 12 is positioned on a retractor support arm 11 by positioning the support arm clamping surface 14 that defines a support arm clamping socket 38 proximate the retractor support arm 11. Manual force is placed upon the bottom clamping member 12 and the support arm 11 such that a constricted entrance 39 to support arm clamping socket 38 opens and the support arm 11 is forced into the clamping socket 38.

Referring to FIGS. 1-4, when the clamp 10 is in the non-clamping position, the bottom clamping member 12 is retained on the retractor support arm 11 by the constricted entrance 39 to the support arm clamping socket 38. In an exemplary embodiment, a bead or raised surface 40 is disposed along lengths of resilient portions 45, 37 where the bead or raised surface 40 constricts the entrance 39. However, other devices that constrict the entrance 39 to the first clamping socket 38 are within the scope of the present invention including, but not limited to, a clip, a roller or a spring-loaded device. As manual force is placed upon the bottom clamping member 12 and the support arm 11, the resilient portions 45, 47 move to open the entrance 39 to the support arm clamping socket 38 thereby allowing the support arm 11 to be positioned within the support arm clamping socket 38.

The constricted entrance 39 provides a preliminary clamping force around the portion of the retractor support arm 11 such that the bottom clamping member 12 does not slip off of the support arm 11 when the clamp 10 is in the non-clamping position. The entrance 39 of the support arm clamping socket 38 should not be so constricted to prevent use of manual force positioned in the bottom clamping member 12 about the portion of the retractor support arm 11. However, the entrance 39 of the support arm clamping socket 38 should be sufficiently constricted to prevent the bottom clamping member 12 from accidentally slipping off of the retractor support arm 11. An exemplary amount of constriction of the entrance 39 of the support arm clamping socket 38 is between about 0.010 inches and 0.020 inches and preferably about 0.015 inches.

The support arm clamping socket 38 permits the bottom clamping member 12 to be placed on the retractor support arm 11 without moving any other surgical equipment having been previously positioned on the retractor support arm 11. By socket is meant an opening or a cavity into which an inserted part such as a retractor support apparatus, is designed to fit and wherein the retractor support apparatus can be inserted into the socket from an infinite number of directions in a 180 degree range starting from a substantially parallel position to a back surface of the socket to a position substantially perpendicular to the back surface and continuing to position again substantially parallel to the back surface of the socket. By opening is meant an entrance to the clamping socket from an external surface of the clamping member into which a portion of a part such as the retractor support apparatus is inserted in a direction that is not parallel to a central axis of the part where the opening may be constricted or non-constricted. Preferably the part is inserted through the opening and into the clamping socket in a direction substantially perpendicular to the central axis of the part, although other directions are within the scope of the present invention.

Referring to FIGS. 2-4, the support arm clamping surface 14 is configured to generally conform to the cross sectional configuration of the retractor support arm 11, but may be configured to other shaped cross sections. In a preferred embodiment, the support arm clamping surface 14 conforms to a substantially circular cross sectional configuration of the retractor support arm 11.

The bottom clamping member 12 is preferably of a unitary construction and includes a fulcrum portion 28 between the clamping end 24 and an attachment end 26. The fulcrum portion 28 extends between an upper leg portion 32 and a lower leg portion 34, which define a recess 30 proximate the attachment end 26. As the upper and lower leg portions 32, 34 are spread apart, the fulcrum portion 28 flexes resulting in a contraction of the support arm clamping socket 38. The contracted support arm clamping socket 38 creates a frictional engagement between the support arm 11 and the support arm clamping surface 14.

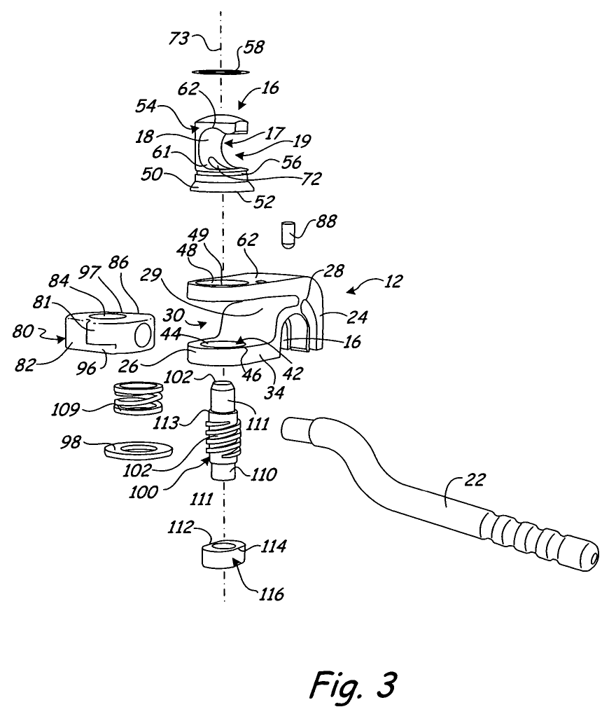

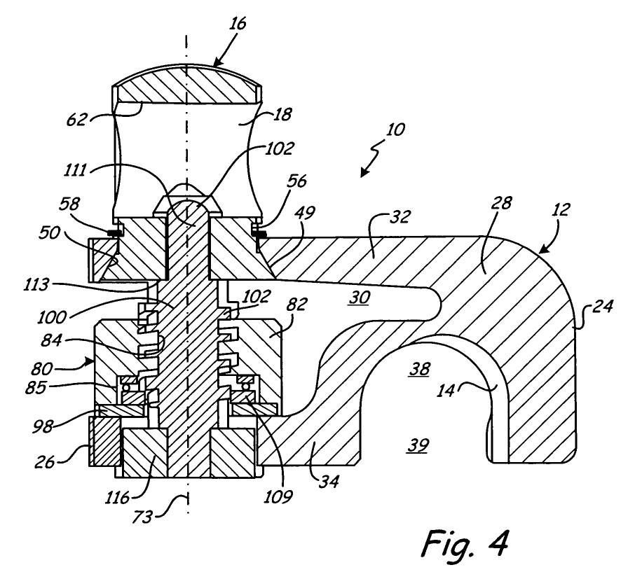

The upper and lower leg portions 32, 34 are spread apart with an actuating mechanism 80 positioned within the recess 30. Referring to FIGS. 3 and 4, the actuating mechanism 80 preferably includes a threaded bore 84 which engages a threaded region 102 of a shaft 100 that is non-rotatably positioned within the recess 30 between the upper and lower leg portions 32 and 34.

Referring to FIGS. 1-4, the actuating mechanism 80 rotates and travels down the threaded region 102 such that the actuating mechanism 80 engages the bottom leg portion 34. Further rotation of the actuating mechanism 80 forces the shaft 100 toward the upper leg portion 32. As the shaft 100 is forced upward, a shoulder 113 on the shaft engages a proximal end 52 of the top clamping member 16.

As the shoulder 113 engages the proximal end 52, the top clamping member 16 is fixed in a selected rotational position by a frictional engagement with the upper leg portion 32 of the bottom clamping member 12. The force from the shoulder 113 causes a frusto-conical surface 50 of the top clamping member 16 to frictionally engage a frusto-conical surface 49 defining a through bore 48 in the upper leg portion 32 and thereby prevents rotation of the top clamping member 16. When the actuating mechanism 80 is positioned in the non-clamping position, the top clamping member 16 rotates within the through bore 48 and is captivated to the upper leg portion 16 with a snap ring 58 positioned within an annular groove 56 about a perimeter of the top clamping member 16.

A retractor handle 20 is manually forced within a retractor clamping socket 17 defined by the retractor clamping surface 18 positioned on a distal end portion 54 of the top clamping member 16 with manual force. An arcuate upper portion 62 of the retractor clamping surface 18 and an end 102 of the shaft 100 positioned through a through bore 72 constrict an entrance 19 of the retractor clamping socket 17. The through bore 72 extends through the top clamping member 16 along an axis 73 and intersects a bottom portion 61 of the retractor clamping surface 18 as best illustrated in FIGS. 3 and 4.

The end 102 of the shaft is biased into the retractor clamping socket 17 with a compression spring 109 positioned about the shaft 100 and between the actuating mechanism 80 and the lower leg portion 34. Manual force on the retractor handle 20 forces the end 102 of the shaft 100 from the retractor clamping socket 17 by compressing the compression spring 109 into a recess 85 in the actuating mechanism 80 which allows the retractor handle 20 to be positioned therein. With the retractor handle 20 positioned within the retractor clamping socket 17, the compression spring 109 biases the end 102 of the shaft 100 back into the retractor clamping socket 17 and thereby slidably retains the retractor handle 20 therein.

Alternatively, the shaft 100 may threadably engage a threaded through bore 42 in the lower leg portion 34. A handle 22 may be fixedly attached to the shaft 100 to rotate the shaft 100 into the clamping position from the non-clamping position. Because the shaft 100 does not move or float along the axis 73, when the clamp 10 is in the non-clamping position the end 102 of the shaft 100 is positioned away from the retractor clamping socket 17. When the shaft 100 is rotated into the clamping position, the end 102 of the shaft 100 is forced into the retractor clamping socket to frictionally retain the retractor handle 20 therein.

Although the top clamping member 16 is described and illustrated in the drawings as retaining and clamping a retractor handle 20, the top clamping member 16 can also retain and clamp other surgical devices. By surgical devices is meant any element that is useful in conducting a surgical procedure including, but not limited to, a retractor support apparatus, a retractor or any other medical instrument that is used during a surgical procedure such as a camera, a light or a catheter.

Prior to using the clamp 10, a position of the shaft 100 within the clamp 10 is adjusted with a screwdriver engaging a slot 111 such that the top and bottom clamps 16, 12, respectively, are in a non-clamping position when the actuating mechanism 80 is in a non-clamping position and the top and bottom clamps 16, 12, respectively, are in a clamping position when the actuating mechanism 80 is in the clamping position. The threaded engagement of the threaded region 112 with the threaded bore 84 adjusts the position of the shaft 100 with respect to the actuating mechanism 80 such that the clamp 10 functions properly.

With the shaft 100 in the selected positioned within the clamp 10, a cap 116 is fixedly attached to the shaft 100, preferably with a weld. The cap 116 has substantially flat, parallel sides 112, 114 that cooperate with substantially flat parallel sides 44, 46, respectively, of an elongated slot 42 in the lower leg portion 34. The engagement of the sides 112, 114 of the cap 116 with the sides 44, 46 of the elongated slot 42 prevent rotation of the shaft 100 within the clamp 10 while allowing the shaft 100 to move along the axis 73 with respect to the bottom and top clamping members 12, 16.

A washer 98, although not necessary to practice the present invention, is positioned between the actuating mechanism 80 and the lower leg portion 34 to prevent either a actuating mechanism 80 or the lower leg portion 34 from wearing over time with use. The washer 98 is preferably made of a wear resistant polymer such as PEEK.

The top clamping member 16 is positioned into a clamping position at approximately the same time that the actuating mechanism 80 forces the upper and lower leg portions 32, 34 apart by positioning the actuating mechanism 80 into the second clamping position. To position the actuating mechanism 80 into the second clamping position, a handle 22 fixedly attached to the actuating mechanism 80 is moved in a direction of arrows 23, as illustrated in FIG. 1, which rotates the actuating mechanism 80 about an shaft 100 and the axis 73. A pitch of threads of the threaded region 102 of the shaft 100 and the threads within the bore 84 of the actuating mechanism 80 are designed such that as the actuating mechanism 80 is rotated about 180 degrees, the clamp 10 is positioned from the first non-clamping position to the second clamping position.

As the actuating mechanism 80 is rotated, the bore 84 threadably engages the threaded region 102 of the shaft 100 and forces the actuating mechanism 80 towards the lower leg portion 34. As the actuating mechanism 80 is forced into the lower leg portion 34, and a force is placed upon the lower leg portion 34, the actuating mechanism 80 and the shaft 100. A maximum force is placed upon the lower leg 34, the actuating mechanism 80 and the shaft 100 when the actuating mechanism 80 is rotated approximately 180 degrees opposite the non-clamping position where an extension 81 from the actuating mechanism 80 contacts a surface 29 defining an inner region of the recess 30 as best illustrated in FIGS. 2 and 3.

Referring to FIG. 4, with the actuating mechanism 80 in the second position, the compression spring 109 is compressed within the recess 85 and the actuating mechanism 80 transfers the force to the lower leg portion 34. The end 102 of the shaft 100 is forced into the retractor clamping socket 17 of the top clamping member 16 and frictionally secures the retractor handle 20 within the retractor clamping socket 17 between the end 102 of the shaft 100 and the arcuate upper portion 62.

Further, with the actuating mechanism 80 in the second position, the shoulder 113 of the shaft 100 is forced into the proximal end 52 of the top clamping member 16 thereby causing a frictional engagement between the frusto-conical surfaces 49, 50 of the top clamping member 16 and the upper leg portion 32. The frictional engagement of the frusto-conical surfaces 49, 50 secures the top clamping member 16 in a selected position with respect to the bottom clamping member 12.

Further, with the actuating mechanism 80 in the second position, the distance between the shoulder 113 and a bottom surface 96 of the actuating mechanism 80 is greater than the distance between the washer 98 and the upper leg portion 32 such that the upper and lower leg portions 32, 34 are forced apart thereby transferring the force to the upper leg portion 32 from the shaft 100. One skilled in the art will recognize that the pitch of threads on the threaded region 102 and the threaded bore 84 can be adapted to increase or decrease the distance of travel of the actuating mechanism 80 on the shaft 100 as is necessary to place the clamp 10 into the clamping position.

With the upper and lower leg portions 32, 34 forced apart, the fulcrum portion 28 flexes which causes the support arm clamping socket 38 to constrict such that the support arm clamping surface 14 frictionally engages the retractor support arm 11.

Referring to FIG. 3, an upper surface 97 of the actuating mechanism 80 includes an indention 86 which engages a pin 88 extending from the upper leg portion 34 and into the recess 30 when the actuating mechanism 80 is in a non-clamping position. The engagement of the pin 88 with the indention 86 retains the actuating mechanism 80 in the first non-clamping position such that the bottom and top clamping members 12, 16 are in non-clamping positions.

One skilled in the art will recognize that a plane of movement of the handle 22 approximately intersects an axis of the retractor support arm 11 as best illustrated in FIG. 1. Therefore, the operator can conveniently manipulate the actuating mechanism 80 from the first non-clamping position to the second clamping position by gripping the handle 22 and the retractor support arm 11 with one hand and forcing the handle 22 towards the retractor support arm 11.

The surgical clamp 10 of the present invention also provides a low profile clamping device for conducting the surgical procedure. What is meant by low profile is that the top clamping member 16 and the handle 22 are disposed proximate the retractor support arm 11. By disposing the top clamping member 16 and the handle 22 proximate the retractor support arm 11, a surgical site remains relatively open and free of obstruction thereby providing better access to the surgical site.

An advantage of the clamp 10 of the present invention is that the clamp 10 does not have to be slid along the retractor support arm 11 to a new selected position. The retractor clamp 10 can be repositioned on the support arm 11 by first positioning the actuating mechanism 80 into the first non-clamping position such that the support arm clamping socket 38 is not constricted. The bottom clamping member 12 is then detached from the retractor support arm 11 with manual force in an opposite direction as used to position the bottom clamping member 12 on the retractor support arm 11. After the clamp 10 has been removed from the retractor support arm 11 the clamp 10 is repositionable on the retractor support arm 11 by positioning the entrance 39 to the support arm clamping socket 38 against the retractor support arm 11 and applying manual force substantially perpendicular to the axis of that portion of the retractor support arm 11.

The retractor handle 20 can also be repositioned within the top clamping member 16 without having to slide the retractor handle 20 with respect to the second clamping surface 18. The retractor handle 20 is removed from the top clamping member 16 by providing manual force in the opposite direction of the force used to position the retractor handle 20 within the retractor clamping socket 17. The retractor handle 20 can be reinserted into the retractor clamping socket 17 by reapplying manual force generally perpendicular to the axis of the retractor handle 20.

The surgical clamp 10 of the present invention having the support arm clamping surface 14 and the retractor clamping surface 18 enables the clamp 10 to be positioned upon the retractor support arm 11 in a selected position. Further, an additional retractor 20 can be easily and conveniently positioned within a surgical site without having to position the end of the retractor through the retractor clamping socket 17 defined by the retractor clamping surface 18. The convenience of the surgical clamp 10 of the present invention allows the surgical site to be quickly assembled, modified during a surgical procedure, and disassembled, which enables a surgical team to conduct a more efficient surgical procedure.

Although the present invention has been described with reference to preferred embodiments, workers skilled in the art will recognize that changes may be made in form and detail without departing from the spirit and scope of the invention.

1. A surgical clamp comprising:
a first clamping member comprising a first clamping socket for engaging a first surgical rod and an attachment end;
a second clamping member rotatably mounted with respect to the first clamping member wherein the second clamping member comprises a second clamping socket for engaging a second surgical rod;
a shaft nonrotatably positioned within the attachment end of the first clamping member and slidable therein and wherein the shaft is in communication with the second clamping member; and
a force providing mechanism positioned between the first and second clamping members and disposed about the shaft wherein when the actuating mechanism is positionable between a first position with the first and second clamping members being in a nonclamping position and a second position with the first and second clamping members being in a clamping position wherein the first and second sockets constrict to retain the first and second surgical rods in selected positions.

2. The surgical clamp of claim 1 wherein the force providing mechanism is rotatably positionable about the shaft.

3. The surgical clamp of claim 1 wherein the force providing mechanism comprises a threaded bore threadably engaging the shaft wherein the force providing mechanism applies a force to the first and second clamping members when positioned into the second position such that the first and second sockets constrict to position the first and second clamping members in the clamping position.

4. The surgical clamp of claim 3 and wherein the attachment end of the first clamping member comprises:
an upper leg; and
a lower leg and wherein the upper and lower legs are forced apart to position the first clamping member into the clamping position.

5. The surgical clamp of claim 1 and wherein the first clamping member further comprises a fulcrum portion between the first clamping socket and the attachment end and wherein the fulcrum portion flexes to constrict the first clamping socket when the first clamping member is in the clamping position.

6. A surgical clamp of claim 1 and wherein the second claming member comprises a through bore and wherein the through bore intersects the second clamping socket and wherein an end of the shaft protrudes in the second clamping socket through the through bore to constrict the second clamping socket when the force providing mechanism is positioned into the second clamping position.

7. The surgical clamp of claim 1 and wherein the second clamping member frictionally engages the first clamping member to fix a rotational position of the second clamping member when the force providing mechanism is positioned into the second clamping position.

8. A surgical clamp comprising:
a clamping portion;
a first leg extending from one side of the clamping portion;
a second leg extending from another side of the clamping portion, the first and second legs being spaced apart;
a shaft extending through the first and second legs; and
an actuating mechanism threadably engaging the shaft between the first and second legs, the actuating mechanism being movable to force the first and second legs to move in opposing directions thereby placing the clamping portion in the clamping position.

9. The surgical clamp of claim 8 wherein the actuating mechanism is rotatably movable between the first and second legs.

10. The surgical clamp of clamp 8 wherein the clamping portion comprises a clamping socket whereby when the first and second legs are moved in opposing directions the clamping socket constricts.

11. The surgical clamp of claim 10 and further comprising a fulcrum portion located between the clamping socket and the first and second legs wherein when the first and second legs are forced apart the fulcrum portion flexes and constricts the clamping socket.

12. The surgical clamp of claim 8 further comprising a handle attached to the actuating mechanism.

13. The surgical clamp of claim 8 and further comprising a second clamping member being rotatably attached to either the first leg or the second leg and wherein the second clamping member being positioned in a clamping position by movement of the actuating mechanism.

14. A surgical clamp for clamping at least two elongated members, each member having a central axis, the clamp comprising:
at least two clamping members, each clamping member having a clamping socket with an opening for accepting an elongated member in a generally orthogonal direction to the axis of the member; and
a mechanism that constricts the opening of the clamping socket of both members generally simultaneously such that the respective elongated member is captivated within each clamping socket.

15. The surgical clamp of claim 14 wherein one of the clamping member comprises first and second spaced apart legs wherein the mechanism forces the first and second spaced apart legs in opposite directions that results in constriction of the opening of the clamping socket of that clamping member.

16. The surgical clamp of claim 14 and further comprising a shaft coupled to the mechanism wherein the shaft is attached to the two clamping members and has an end that extends into the clamping socket of one of the two clamping members to place that clamping member in a clamping position.

17. The surgical clamp of claim 16 wherein the mechanism includes a threaded bore that threadably engaging the shaft and wherein as the mechanism threadably rotates about the shaft a force is placed upon the two clamping members to constrict the clamping sockets.
